(19) United States
(12) Patent Application Publication
(10) Pub. No.: US 2009/0131789 A1
(43) Pub. Date: May 21, 2009

(54) DEVICE FOR X-RAY BRACHYTHERAPY, AND METHOD FOR POSITIONING A PROBE INTRODUCED INTO A BODY FOR X-RAY BRACHYTHERAPY

(76) Inventors: Jens Fehre, Hausen (DE); Bernd Granz, Oberasbach (DE); Markus Lanski, Wasseralfingen (DE); Ralf Nanke, Neunkirchen am Brand (DE)

Correspondence Address: SCHIFF HARDIN, LLP;PATENT DEPARTMENT, 6600 SEARS TOWER, CHICAGO, IL, 60606-6473, US

(21) Appl. No.: 12/094,933

(22) PCT Filed: Sep. 28, 2006
(86) PCT No.: PCT/EP2006/066845

§ 371 Date: Oct. 23, 2008

(30) Foreign Application Priority Data
Nov. 24, 2005 (DE) 10 2005 056 067.9

(51) Int. Cl. A61B 8/00; A61N 5/10

(52) U.S. Cl. 600/439; 378/65

(57) ABSTRACT

In a method and a device for x-ray brachytherapy, a probe is inserted into the interior of the body of a subject, the probe carrying at its distal end an x-ray source that radiates x-rays into an exposure area in the body of the subject outside of the probe. The probe also contains an x-ray array that generates an ultrasound image representing at least a portion of the exposure area. A display device displays the ultrasound image to allow identification of the exposure area in the ultrasound image.

### BACKGROUND OF THE INVENTION

1. Field of the Invention

The present invention concerns a device for x-ray brachytherapy as well as a method for positioning of a probe inserted into the inside of a body for x-ray brachytherapy.

2. Description of the Prior Art

X-ray brachytherapy is a therapeutic treatment with x-rays in which the x-ray source is brought very close to the tissue to be treated (for example a tumor or a vessel wall) after implementation of a endovascular dilatation. In order to able to insert the x-ray source with the aid of a catheter or a probe either without an invasive procedure or with an optimally minimally-invasive procedure inside a body, a miniaturized x-ray source is required as is known from U.S. Pat. No. 6,721,392, for example. This is arranged at the distal end of a probe that, for example, is intraoperatively positioned in a tumor or tumor bed (after its extraction), as explained in detail in the PR information of Carl Zeiss A G, Medical Engineering Innovation by Carl Zeiss A G, “Intraoperative Strahlentherapie mit dem INTRABEAM System von der Carl Zeiss A G” state as of September 2004, for example.

A miniaturized x-ray source that is arranged in a catheter with which it can be inserted into the body cavities (lumen) in order to irradiate selected tissue zones from the immediate surroundings is known from United States Patent Application Publication—2003/0149327. The x-ray source has a shielding rotatable around the axis of the catheter in order to radiate the x-rays in a targeted manner at least perpendicular to the axis in a selected solid angle. The environment of the catheter can be observed with an optical observation device arranged in a catheter. A light source that exposes only the part of the surface of the hollow space that is also irradiated is used for this purpose.

In endovascular brachytherapy with a beta or gamma radiator arranged in the tip of a catheter, it is also known from DE 10 2004 008 373 B3 (for example) to arrange an optical observation device in the catheter. For this purpose, a brachytherapy catheter is integrated into a unit with an OCT catheter operating on the basis of optical coherence tomography (OCT).

For the therapeutic success it is essential that the x-rays radiating out of the catheter from the x-ray source in an exposure area for most part exclusively strike the tissue (for example the tumor) to be treated in order to ensure an optimally low exposure of the healthy tissue located near this. This requires a precise positioning of the exposure area, i.e. a precise positioning and alignment of the x-ray source or, respectively, of the solid angle in which the x-rays exit.

### SUMMARY OF THE INVENTION

An object of the invention is provide a device for x-ray brachytherapy with a probe that can be inserted inside a body, with which probe a precise positioning of the exposure area is possible. A further object of the invention is to provide a method for positioning of a probe inserted inside a body for x-ray brachytherapy.

With regard to the device, the above object is achieved by a device that contains a probe that can be inserted into the inside of a body, the probe at its distal end having an x-ray source that radiates x-rays into an exposure area outside of the probe, wherein an ultrasound array for generation of an ultrasound image rendering at least a portion of the exposure area is arranged in the probe. This enables a precise positioning of the exposure area, i.e. a precise positioning of the x-ray source and of the solid angle range in which the x-rays generated by the x-ray source are radiated.

As used herein the term “probe” is an instrument that can be introduced into the inside of a body. This can be both a catheter (in the narrower sense) that is inserted into body cavities (transluminal) and a needle-like instrument that can be placed within a tissue zone (percutaneous or interstitial).

If a shielding that can be moved relative to the x-ray source is arranged in the probe to adjust the exposure area, the exposure area can be adjusted more flexibly even given a stationary probe.

In a preferred embodiment of the invention, the device has a display device to show the ultrasound image and to identify the exposure area in the ultrasound image. This enables a particularly simple and graphic positioning of the exposure area.

Moreover, if lines of equal dose rating are mixed into the exposure area rendered in the ultrasound image, the dose rating required at different locations of the exposure area can be adjusted in a targeted manner.

In a further preferred embodiment, the position of the x-ray source is also displayed in the ultrasound image.

It is advantageous when an optical image of a surroundings of the probe containing at least a portion of the exposure area is generated in addition to the ultrasound image with the aid of an optical observation device arranged in or on the probe. The correct navigation of the probe is then in particular additionally made easier when the exposure area is identified in the optical image.

A device according to the invention is suitable for insertion into a urethra or a ureter and for therapeutic treatment of a prostate, bladder or kidney tumor.

The above object also is achieved in accordance with the present invention by a method for positioning a probe inserted into the interior of the body of a subject for x-ray brachytherapy, the probe having an x-ray source at a distal end thereof that radiates x-rays into an exposure area in the body of the patient outside of the probe, the method including generating an ultrasound image that shows at least a portion of the exposure area with an ultrasound array located in the probe, with the ultrasound image being used to correctly position the exposure area within the body of the subject.

The aforementioned advantages associated with the inventive device are also achieved by the inventive method.

In an embodiment of the method, an optical image of a subject region containing at least one part of the exposure area is generated in which optical image the exposure area is indicated, in particular in that a luminophore is applied that preferably accumulates in a tumor. The exposure area can then be correctly positioned in a particularly simple and graphic manner in that the exposure area is brought into congruence with the area marked by the luminophore.

The method according to the invention is suitable in the treatment of a prostate, bladder or kidney tumor in which the probe is inserted into a urethra or a ureter.

### BRIEF DESCRIPTION OF THE DRAWINGS

FIG. 1 schematically illustrates a device for x-ray brachytherapy constructed and operating in accordance with the present invention.

FIG. 2 illustrates an ultrasound image obtained in accordance with the present invention, in which the exposure area is displayed.

FIG. 3 illustrates an optical image of the environment of the probe obtained in accordance with the present invention, in which the exposure area is identified.

### DESCRIPTION OF THE PREFERRED EMBODIMENTS

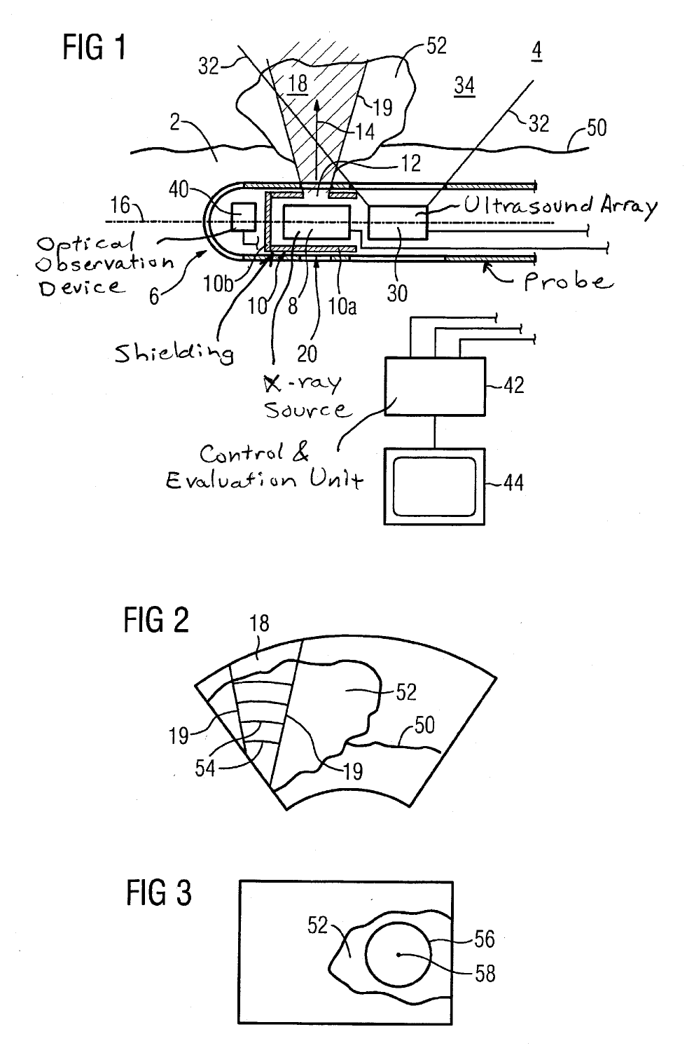

According to FIG. 1, a probe 6 (a catheter in the example) in which an x-ray source 8 is arranged at its distal end is inserted into a cavity (lumen) 2 of a body 4 (which can be a urethra or a ureter). A shielding 10 is associated with the x-ray source 8, which shielding 10 in the exemplary embodiment contains a cylindrical part 10a that is provided in its circumference with a diaphragm or aperture 12 through which x-rays 14 can exit perpendicular to the longitudinal axis 16 of the probe 6, i.e. radially in a (for example conical) exposure area 18 defined by the shape of the aperture 12 and its distance from the anode of the x-ray source 8, which exposure area 18 is emphasized by hatching in the Figure and is indicated by boundary lines 19.

The cylindrical part 10a of the shielding 10 is arranged within the probe 6 such that it can be rotated around its longitudinal axis 16 so that the exposure area 18 can likewise be pivoted on this longitudinal axis 16. For this purpose, the outer wall of the probe 6 is provided with a window 20 rotating annularly, which window 20 is permeable to x-rays 14. However, the probe 6 can also be permeable to x-rays 14 in a larger region and, for example, possess an outer wall consisting of a polymer material.

The shielding 10 has on its front side a front plate 10b that is provided with a closable diaphragm (not shown in detail in FIG. 1) with which it is possible to selectively radiate x-rays 14 in the direction of the longitudinal axis 16. In this case either a movable lock is provided with which the aperture 12 can be closed or an additional shielding is provided that is arranged in the probe 6 such that the aperture 12 can be positioned in the region of this shielding. For example, this can be realized in that the window 20 does not extend over its entire extent.

An ultrasound array 30 that generates an ultrasound image of a subject region 34 (illustrated by boundary lines 32) that overlays at least a portion of the exposure area 18 is arranged in the immediate proximity of the x-ray source 8.

Moreover, the probe 6 contains a schematically indicated optical observation device 40 with which a subject region can be observed that renders at least a portion of the exposure area 18.

The probe 6 is connected to a control and evaluation device 42 with which the x-ray source 8, the ultrasound array 30 and the observation device 40 are controlled and the signals transmitted from the ultrasound array 30 and from the observation device 40 are evaluated so that they can be shown as an ultrasound image or as an optical image on a display device 44 (for example a monitor).

In the example of FIG. 1 a tissue zone 52 to be therapeutically treated (for example a tumor, in particular a prostate tumor, a bladder tumor or a kidney tumor that should be irradiated with x-rays 14) is located in the region of the wall 50 of the cavity 2.

This tissue zone 52 as well as the wall 50 are schematically depicted in the ultrasound image of FIG. 2. In this ultrasound image the intersection surface of the exposure area 18 with the subject plane detected by the ultrasound array is, for example, identified by rendering its lateral boundary lines 19. The exposure area 18 mixed into the ultrasound image in this manner enables a correct positioning of the probe or, respectively, of the x-ray source.

Moreover, approximately circular lines 54 of equal dose rating that indicate the current local dose rating to the therapist are mixed into the ultrasound image, for instance. These lines 54 are located, for example, located among one another at an interval that corresponds to the decrease of the dose rating to 1/e, respectively. These are approximately 1.2 cm given an x-ray radiator with an average energy of 20 keV in the tissue. The therapist can then adjust the required dose rating by changing the operating parameters of the x-ray source (anode current, acceleration voltage). These lines 54 of the same dose rating then shift in the ultrasound image corresponding to the dose rating changing with the variation of the operating parameters.

According to FIG. 3, an optical image in which the wall 50 of the cavity is visible given corresponding illumination is generated from the surroundings of the probe. If a luminophore is additionally applied (for example directly with the aid of the probe) which preferably accumulates in the tissue zone 52 if this is a tumor and that, for example, is excited by the light source used for illumination and emits fluorescence light in the visible range, the tumor 52 can also be made visible in the optical image at least in its surface region with which it borders the cavity. If the envelope if the x-ray beam striking the surface of the wall 50 is additionally mixed in as a circular line 56 and its center axis is mixed in as a point 58, the exact positioning can also be effected with the aid of the optical image in addition to the positioning with the ultrasound image since in this case image information exists from a different plane, for example a plane perpendicular to the image plane of the ultrasound image.

The invention is described using a catheter inserted into the cavity of a body. In principle the invention is also suitable for probes that are directly inserted into the tissue, as this is the case in the invasive post-treatment (explained above) of a tumor bed of a previously removed tumor. The tissue zone to be treated can also be a vessel wall that should be irradiated after the implementation of a dilatation to reduce the restenosis rate.

Although modifications and changes may be suggested by those skilled in the art, it is the intention of the inventors to embody within the patent warranted heron all changes and modifications as reasonably and properly come within the scope of their contribution to the art.

We claim as our invention:

1-18. (canceled)

19. A device for x-ray brachytherapy comprising:
a probe configured for insertion into the body of a living subject, said probe having a distal end;
an x-ray source located in the probe at the distal end thereof that radiates x-rays for x-ray brachytherapy into an exposure area in the body of the subject outside of the probe;
an ultrasound array located in the probe that generates an ultrasound image representing at least a portion of the exposure area; and
a display device connected to the ultrasound array that visually displays the ultrasound image to allow identification of the exposure area in the display ultrasound image.

20. A device as claimed in claim 19 comprising a shielding in said probe that is movable relative to the x-ray source to adjust a position of the exposure area in the body of the subject.

21. A device as claimed in claim 19 comprising a mixing device that mixes lines of equal x-ray dose rating into the exposure area displayed in the ultrasound image.

22. A device as claimed in claim 19 wherein said display displays the position of the x-ray source in the ultrasound image.

23. A device as claimed in claim 19 comprising an optical observation device contained in the probe that generates an optical image of an environment of the probe in the body of the subject, encompassing at least a portion of the exposure area.

24. A device as claimed in claim 23 comprising a display device at which said optical image is displayed, said display device that displays said optical image identifying the exposure area in the optical image.

25. A method for x-ray brachytherapy comprising:
introducing a probe into the body of a living subject, said probe having a distal end;
from an x-ray source located in the probe at the distal end thereof, radiating x-rays for x-ray brachytherapy into an exposure area in the body of the subject outside of the probe;
with an ultrasound array located in the probe, generating an ultrasound image representing at least a portion of the exposure area; and
visually displaying the ultrasound image to allow identification of the exposure area in the display ultrasound image.

26. A method as claimed in claim 25 comprising adjusting the exposure area by moving a shielding in the probe relative to the x-ray source.

27. A method as claimed in claim in 25 comprising mixing lines of equal x-ray dose rating into the exposure area in the display of the exposure area in the ultrasound image.

28. A method as claimed in claim 25 comprising identifying a position of the x-ray source in the displayed ultrasound image.

29. A method as claimed in claim 25 comprising providing an optical observation device in the probe and, with said optical observation device, generating an optical image of an environment of the probe that encompasses at least a portion of the exposure area, and displaying said optical image.

30. A method as claimed in claim 29 comprising identifying the exposure area in the displayed optical image.

31. A method as claimed in claim 29 comprising applying a luminophore to tissue in said environment of the probe that preferentially accumulates in tumorous tissue, to cause said tumorous tissue to be visible in the displayed optical image.

32. A method as claimed in claim 29 comprising introducing the probe into the body of the subject through the urethra or the ureter of the subject.

33. A method as claimed in claim 33 comprising implementing said x-ray brachytherapy to treat tumorous tissue selected from the group consisting of tumorous prostate tissue, tumorous bladder tissue, and tumorous kidney tissue.
